(19) United States
(12) Patent Application Publication
(10) Pub. No.: US 2011/0105818 A1
(43) Pub. Date: May 5, 2011

(54) Dehydrogenation Catalyst with a Water Gas Shift Co-Catalyst

(75) Inventors: Joseph E. Pelati, Huoston, TX (US); Hollie Craig, Seabrook, TX (US); James R. Butler, League City, TX (US)

(73) Assignee: Fina Technology, Inc., Houston, TX (US)

(21) Appl. No.: 12/610,310

(22) Filed: Oct. 31, 2009

(51) Int. Cl. C07C 5/333; B01J 23/10; B01J 27/232; B01J 21/18; B01J 31/02; B01J 27/236; B01J 27/25

(52) U.S. Cl. 585/444; 585/440; 502/304; 502/174; 502/184; 502/159; 502/176; 502/201

(57) ABSTRACT

A catalyst comprising a dehydrogenation catalyst and a water gas shift co-catalyst can be used for the dehydrogenation of alkylaromatic hydrocarbons to alkenylaromatic hydrocarbons. For instance, the catalyst can be used for the dehydrogenation of ethylbenzene to styrene. The catalyst can include an iron compound, a potassium compound, and a cerium compound.

### CROSS-REFERENCE TO RELATED APPLICATIONS

Not applicable.

### FIELD

The present invention generally relates to a dehydrogenation catalyst for the dehydrogenation of alkylaromatic hydrocarbons to alkenylaromatic hydrocarbons.

### BACKGROUND

Dehydrogenation catalysts can be used for the conversion of alkylaromatic hydrocarbons to alkenylaromatic hydrocarbons. These catalysts can be used, for instance, in the dehydrogenation of ethylbenzene to styrene. Styrene is the monomer from which the polymer polystyrene is formed. Polystyrene is a well-known plastic with many commercial uses. It can be extruded, injection molded, or blow molded to make objects like plastic utensils and casings for CDs. Polystyrene can also be formed with a rubber component, such as polybutadiene, to make high impact polystyrene, or HIPS. It is also commonly used in a foamed form.

Conventional dehydrogenation catalysts typically comprise an iron oxide, a potassium source, and optionally a cerium source. Other elements, which can act as stabilizers and/or promoters, including Cr, Mo, W, Ca, Na, and others can also be included in the composition of the dehydrogenation catalyst.

Dehydrogenation catalysts can be evaluated in terms of conversion, selectivity, and lifetime. Conversion (or activity) generally refers to the portion or percentage of feed hydrocarbons that are converted into product hydrocarbons. Selectivity generally refers to the portion of all product hydrocarbons that a certain desired product comprises. A catalyst's activity usually decreases over time, eventually ending in breakdown or deactivation of the catalyst. A catalyst's lifetime is the time that a catalyst can be used in a dehydrogenation reactor until breakdown or deactivation of the catalyst necessitates its regeneration or replacement. Regeneration and replacement are processes that can be expensive due to the lost production during replacement and/or the expenses involved in regenerating the catalyst. Any increase in stability of the catalyst that would promote a longer catalyst life would enhance the economics of the process using the catalyst.

Catalyst deactivation can be caused by several factors, one of which is coking Coking is the carbonization of the catalyst surface wherein pores on the catalyst surface are physically plugged by carbonaceous deposits (coke). Coking can decrease conversion and selectivity of the catalyst and can necessitate an undesirable frequency of catalyst regeneration and/or replacement.

One method of de-coking, in which the catalyst is regenerated, involves the steaming and heating of the catalyst. However, these regenerative operations can lead to the physical breakdown of the catalyst structure. Potassium is a common component of dehydrogenation catalysts. Potassium, however, can be mobile at high temperatures, especially with steam. In the steam de-coking process potassium movement and loss can be a problem, which can be further compounded by any physical breakdown of the catalyst structure.

Thus, it is desirable to prevent coking of the catalyst surface, in order to increase the catalyst's life, to decrease the frequency of regeneration, and to avoid the large amounts of steam used during regeneration. A need exists for a dehydrogenation catalyst whose composition is such that it can prevent coking and help with de-coking.

### SUMMARY

Embodiments of the present invention generally include a catalyst comprising a dehydrogenation catalyst and a water gas shift co-catalyst and a method for using said catalyst in the conversion of alkylaromatic hydrocarbons to alkenylaromatic hydrocarbons.

The catalyst can include 20 to 70 weight percent of an iron compound, 1 to 40 weight percent of an alkali metal compound, 0.5 to 25 weight percent of a cerium compound, and 20 to 80 weight percent of a water gas shift co-catalyst. Optionally, the catalyst can include 25 to 50 weight percent of an iron compound, 10 to 30 weight percent of an alkali metal compound, 5 to 20 weight percent of a cerium compound, and 20 to 50 weight percent of water gas shift co-catalyst.

The iron compound can be an iron oxide or a potassium ferrite. The alkali metal compound can be selected from the group consisting of an alkali metal oxide, nitrate, hydroxide, carbonate, bicarbonate, and combinations thereof, and can comprise a sodium or potassium compound. The alkali metal compound can be a potassium ferrite. The cerium compound can be a cerium oxide. The water gas shift co-catalyst can be any known in the art, for example, a water gas shift catalyst that includes copper oxide, zinc oxide, and alumina.

Optionally, the catalyst can further include 0.1 ppm to 1000 ppm of a noble metal. Optionally the catalyst can further include from an effective amount up to 10 weight percent of at least one non-oxidation catalytic compound chosen from the group consisting of Groups IA, IB, IIA, IB, IIIA, VB, VIB, VIIB and VIII of the periodic table and the rare earth metals. Optionally, the catalyst can further include at least one compound, to enhance physical properties, chosen from the nonexclusive group consisting of graphite, methyl cellulose, and cement.

An embodiment of the invention is a method for the dehydrogenation of alkylaromatic hydrocarbons to alkenylaromatic hydrocarbons. The method includes providing a dehydrogenation catalyst having 20 to 70 weight percent of an iron compound, 1 to 40 weight percent of an alkali metal compound, 0.5 to 25 weight percent of a cerium compound, and 20 to 80 weight percent of a water gas shift co-catalyst to a dehydrogenation reactor. A hydrocarbon feedstock of alkylaromatic hydrocarbons and steam is supplied to the dehydrogenation reactor. The hydrocarbon feedstock and steam are contacted with the dehydrogenation catalyst within the reactor under conditions effective to dehydrogenate at least a portion of said alkylaromatic hydrocarbons to produce alkenylaromatic hydrocarbons. A product of alkenylaromatic hydrocarbons is recovered from the dehydrogenation reactor.

The alkylaromatic hydrocarbons in the feedstock can include ethylbenzene and the alkenylaromatic hydrocarbons of the product can include styrene.

### DETAILED DESCRIPTION

The present invention is for a catalyst that includes a dehydrogenation catalyst and a water gas shift (WGS) co-catalyst. The catalyst can be used in the conversion of alkylaromatic hydrocarbons to alkenylaromatic hydrocarbons, such as the conversion of ethylbenzene to styrene. The catalyst can exhibit improved properties, such as coke prevention, enhanced de-coking, and increased stability.

The catalyst of the present invention can have at least 40% by weight of a conventional dehydrogenation catalyst. Conventional dehydrogenation catalysts generally include an iron compound, an alkali metal compound, and optionally a cerium compound, each comprising certain percentages of the weight of the entire catalyst. All weight percentages given in this application refer to the entire catalyst, including the WGS co-catalyst, and not solely the dehydrogenation co-catalyst.

The iron compound can be in the form of an iron oxide, such as red iron oxide (Fe₂O₃) or yellow iron oxide (FeO(OH)) iron oxide hydrate. Other iron oxides that can be used in accordance with the invention include, but are not limited to, black iron oxides such as magnetite, brown iron oxides such as maghemite, and other yellow iron oxides such as goethite. The iron compound can optionally be a potassium ferrite. Iron oxides used in the invention can be derived from a variety of precursor materials, both natural and synthetic, using any process known in the art. The catalyst of the present invention can contain from 20% to 70% by weight, optionally from 25% to 50% by weight of the iron compound.

The alkali metal compound can be selected from the group consisting of an alkali metal oxide, nitrate, hydroxide, carbonate, bicarbonate, and combinations thereof, and can include a sodium or potassium compound. The alkali metal compound can be a potassium ferrite. The catalyst of the present invention can contain from 1% to 40% by weight, optionally from 10% to 30% by weight of the alkali metal compound.

The cerium compound can be used in the form of cerium oxide. The source for the cerium compound can be a salt that can decompose to the oxide form during calcination, such as a hydrate, carbonate, nitrate, sulfate, or other similar source. The catalyst of the present invention can contain from 0.5% to 25% by weight, optionally from 5% to 20% by weight of the cerium compound.

Other compounds can also be included in the catalyst of the present invention. Such compounds can impart certain properties to the catalyst, such as increasing activity and/or stability of the catalyst. For instance, the catalyst can include 0.1 ppm to 1000 ppm of a noble metal compound, optionally from 1.0 ppm to 800 ppm, optionally from 1.0 ppm to 500 ppm. Additionally, the catalyst can include up to 10% by weight of any of the non-oxidation catalytic compounds of Groups IA, IB, IIA, IB, IIIA, VB, VIB, VIIB and VIII and rare earth metals, such as calcium carbonate, magnesium oxide, chromium or copper salts, or the oxides of elements such as chromium, manganese, aluminum, vanadium, magnesium, thorium and/or molybdenum. These can be added in an effective amount that can vary depending on the compound and its effect on the final catalyst usage. An effective amount can in some instances be at least 0.001 wt %, optionally at least 0.01 wt %, optionally at least 0.1 wt %, optionally at least 1.0 wt %. Other additives, such as carbon black or graphite, methyl cellulose, and cement, can also be included. Additives such as graphite and methyl cellulose can be included within the catalyst mix and can then be burned out during a calcination step, resulting in voids and effecting the porosity of the final catalyst.

The WGS co-catalyst can be any WGS catalyst known in the art, including any low temperature shift catalyst or high temperature shift catalyst. High temperature shift catalysts generally include oxides of iron and chromium, while low temperature shift catalysts generally include copper oxide, zinc oxide, and alumina. One embodiment of a WGS catalyst can generally consist of 45% CuO, 45% ZnO and 10% alumina, however, other combinations of compounds that can be used to form a WGS catalyst are known in the art. A WGS catalyst having a zirconium oxide support that supports one or more of ruthenium, platinum, cobalt, and molybdenum is one non-limiting example. Embodiments including noble metals can generally have up to 5% noble metal loading on the support. Embodiments including transition metal oxides can generally have up to 20% transition metal oxide loading on the support.

The WGS co-catalyst can make up from 5% to 90% by weight of the entire catalyst. Optionally the WGS co-catalyst can make up from 10% to 70% or from 25% to 50% by weight of the entire catalyst.

Carbon monoxide can form in a dehydrogenation reactor from the water gas reaction (Equation 1), wherein carbonaceous deposits, or coke, and steam react to form carbon monoxide and hydrogen. A WGS catalyst catalyzes the water gas shift reaction (Equation 2), wherein carbon monoxide and steam react to form carbon dioxide and hydrogen. The two reactions are shown below.

The Water Gas Reaction: C (coke)+H₂O-->CO+H₂ Equation 1

The Water Gas Shift Reaction: CO+H₂O-->CO₂+H₂ Equation 2

The WGS co-catalyst can help to de-coke the catalyst by removing the carbon monoxide product of equation 1, upsetting the equilibrium of Equation 1 and causing Equation 1 to occur to a greater extent, thereby removing more coke from the catalyst surface.

The catalyst of the present invention can be formed by combining a prepared WGS co-catalyst with the iron, alkali metal, cerium, and other compounds that make up the dehydrogenation catalyst. The WGS co-catalyst can be obtained commercially or prepared by any method known in the art for preparing a WGS shift catalyst. The prepared WGS co-catalyst can then be combined with the compounds of the dehydrogenation catalyst by any known means, including co-precipitation, decomposition, impregnation and mechanical mixing. The mixed catalyst can then be wetted and shaped into any form suitable for placement inside a reactor. Shaping can be done by hand, or by using an extruder or die. The shaped catalyst can then be dried and calcined, such as by any method known in the art. Drying can take place at a temperature of from 100° C. to 200° C. and can last for an hour to several hours. Calcining can take place at temperatures of from 500° C. to 900° C., optionally from 600° C. to 800° C. The calcining temperature can be reached by ramping up the temperature in increments of 50° C. or so over time.

One non-limiting embodiment of the present invention is a method for the dehydrogenation of alkylaromatic hydrocarbons to alkenylaromatic hydrocarbons, for example, the dehydrogenation of ethylbenzene to styrene. The method includes providing a catalyst having at least 20%, or 30%, or 40% by weight of a conventional dehydrogenation catalyst and from 20% to 60% by weight of a water gas shift co-catalyst to a dehydrogenation reactor. A hydrocarbon feedstock of alkylaromatic hydrocarbons and steam can be supplied to the dehydrogenation reactor. The hydrocarbon feedstock and steam can be contacted within the reactor under conditions effective to dehydrogenate at least a portion of said alkylaromatic hydrocarbons to produce alkenylaromatic hydrocarbons. A product of alkenylaromatic hydrocarbons is recovered from the dehydrogenation reactor.

The dehydrogenation reaction can take place according to a set of reaction conditions, which include feedstock specifications, temperature, pressure, and space velocity. Generally these conditions are known in the art, below are some non-limiting examples.

The feedstock, as previously stated, can include an alkylaromatic hydrocarbon and steam. An alkylaromatic hydrocarbon generally has an aromatic group, such as benzene or napthalene, substituted with one or more alkyl groups, wherein the alkyl group is the dehydrogenatable component of the hydrocarbon. The alkyl group can have at least two carbon atoms. Some non-limiting examples of alkylaromatic hydrocarbons that can be used in the present invention include ethylbenzene, cumene, ethyltoluene, di-ethylbenzene and others. Steam can act as a diluent and a heat source and can inhibit the formation of carbonaceous deposits, or coke, on the catalyst surface. Steam and hydrocarbons can be co-injected into the reactor or separately injected. In embodiments the steam and hydrocarbons can be introduced to the reactor in a steam to hydrocarbon molar ratio (also known as a steam to oil, or S/O, ratio) of from 2 to 20, optionally from 3 to 9.

Dehydrogenation reactions are generally endothermic, and the temperature in the reactor can be from 500° C. to 700° C., optionally from 520° C. to 680° C., optionally from 540° C. to 660° C. The pressure can be above atmospheric or sub-atmospheric, such as from 0.3 atm to 1.5 atm. Contact time is generally related in terms of liquid hourly space velocity (LHSV), which is defined as the volume of liquid hydrocarbon reactant per volume of catalyst per hour. An example of a suitable LHSV can be from 0.1 hr⁻¹ to 5 hr⁻¹, but is not limiting herein.

The reactor can be any kind known in the art and can include an inlet or inlets for hydrocarbon and steam and an outlet for product hydrocarbons. For the dehydrogenation of ethylbenzene, the desired product hydrocarbon is styrene. Other hydrocarbons, including benzene and toluene, can also be products.

### EXAMPLE

A catalyst was prepared as an example of one of many embodiments of the present invention. For its preparation, a rudimentary WGS catalyst was formed by intimately mixing a low surface area alumina with CuO and ZnO in a stirred ball mill to sub-micron particles. The WGS catalyst was then calcined at 775° C. This WGS co-catalyst was then mixed with iron oxide hydrate, potassium carbonate, cerium carbonate, molybdenum oxide, calcium carbonate, and cement. The powder mixture was wetted and formed into catalyst particles, and then calcined at 775° C. The final catalyst contained 23.1% by weight of the WGS co-catalyst, 36.2% by weight of iron oxide hydrate, 19.1% by weight of potassium carbonate, 13.2% by weight of cerium carbonate, 1% by weight of molybdenum oxide, 3.4% by weight of calcium carbonate, and 4% by weight of cement.

In a 3-bed adiabatic reactor at 0.37 LHSV, 7.6 PSIA outlet and 8:1 S/O, the catalyst showed 62% conversion and 93 mol % selectivity to styrene.

The above example demonstrates one possible embodiment of the present invention. Several embodiments of the present invention are possible, and not all possible embodiments are explicitly exemplified in this disclosure, but are intended to still be within the scope of the present invention.

The term “alkyl” refers to a functional group or side-chain that consists solely of single-bonded carbon and hydrogen atoms, for example a methyl or ethyl group.

The term “effective amount” can vary depending on the compound and its effect on the final catalyst usage. An effective amount can in some instances be at least 0.001 wt %, optionally at least 0.01 wt %, optionally at least 0.1 wt %, optionally at least 1.0 wt %.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Other and further embodiments, versions and examples of the invention may be devised without departing from the basic scope thereof and the scope thereof is determined by the claims that follow.

What is claimed is:

1. A catalyst for the dehydrogenation of hydrocarbons comprising:
a dehydrogenation catalyst; and
a water gas shift co-catalyst.

2. The catalyst according to claim 1, wherein the dehydrogenation catalyst comprises an iron compound, an alkali metal compound, and a cerium compound.

3. The catalyst according to claim 2, wherein the alkali metal compound is a potassium compound.

4. The catalyst according to claim 1, comprising 20 to 70 weight percent of an iron oxide.

5. The catalyst according to claim 1, comprising 1 to 40 weight percent of an alkali metal compound.

6. The catalyst according to claim 1, comprising 0.5 to 25 weight percent of cerium oxide or cerium carbonate.

7. The catalyst according to claim 1, comprising 0.1 ppm to 1000 ppm of a noble metal.

8. The catalyst according to claim 1, comprising from an effective amount up to 10 weight percent of at least one nonoxidation catalytic compound chosen from the group consisting of Groups IA, IB, IIA, IB, IIIA, VB, VIB, VIIB and VIII of the periodic table and the rare earth metals.

9. The catalyst according to claim 1, comprising an effective amount of at least one compound chosen from the group consisting of graphite, methyl cellulose, and cement added for physical properties.

10. The catalyst according to claim 1, comprising 20 to 80 weight percent of the water gas shift co-catalyst.

11. A catalyst for the dehydrogenation of alkylaromatic hydrocarbons to alkenylaromatic hydrocarbons comprising:
20 to 70 weight percent of an iron compound;
1 to 40 weight percent of an alkali metal compound;
0.5 to 25 weight percent of a cerium compound; and
20 to 50 weight percent of a water gas shift co-catalyst.

12. The catalyst according to claim 11, used in a reaction where the alkylaromatic hydrocarbons comprise ethylbenzene and the alkenylaromatic hydrocarbons comprise styrene.

13. The catalyst according to claim 11, comprising 25 to 50 weight percent of an iron compound, 10 to 30 weight percent of an alkali metal compound, 5 to 20 weight percent of a cerium compound, and 20 to 50 weight percent of a water gas shift co-catalyst.

14. The catalyst according to claim 11, wherein the iron compound is selected from the group consisting of red iron oxides, yellow iron oxides, black iron oxides, brown iron oxides, potassium ferrites, and combinations thereof.

15. The catalyst according to claim 11, wherein the alkali metal compound is selected from the group consisting of alkali metal oxides, nitrates, hydroxides, carbonates, bicarbonates, and combinations thereof.

16. The catalyst according to claim 11, wherein the alkali metal compound is a sodium compound.

17. The catalyst according to claim 11, wherein the alkali metal compound is a potassium compound.

18. The catalyst according to claim 11, wherein the alkali metal compound comprises a potassium ferrite.

19. The catalyst according to claim 11, wherein the water gas shift co-catalyst comprises one or more of aluminum, zinc, or copper.

20. The catalyst according to claim 11, further comprising 0.1 ppm to 1000 ppm of a noble metal.

21. The catalyst according to claim 11, further comprising from an effective amount up to 10 weight percent of at least one non-oxidation catalytic compound selected from the group consisting of Groups IA, IB, IIA, IB, IIIA, VB, VIB, VIIB and VIII of the periodic table and the rare earth metals.

22. The catalyst according to claim 11, further comprising an effective amount of at least one compound selected from the group consisting of graphite, methyl cellulose, and cement.

23. A method for the dehydrogenation of alkylaromatic hydrocarbons to alkenylaromatic hydrocarbons comprising:
providing to a dehydrogenation reactor a catalyst comprising:
20 to 70 weight percent of an iron compound;
1 to 40 weight percent of an alkali metal compound;
0.5 to 25 weight percent of a cerium compound; and
20 to 50 weight percent of a water gas shift co-catalyst;
supplying a hydrocarbon feedstock comprising alkylaromatic hydrocarbons and steam to the dehydrogenation reactor;
contacting the hydrocarbon feedstock and steam with the catalyst within the reactor under conditions effective to dehydrogenate at least a portion of said alkylaromatic hydrocarbons to produce alkenylaromatic hydrocarbons; and
recovering a product of alkenylaromatic hydrocarbons from the dehydrogenation reactor.

24. The method according to claim 23, wherein the feedstock of alkylaromatic hydrocarbons comprises ethylbenzene and the product of alkenylaromatic hydrocarbons comprises styrene.

25. The method according to claim 23, wherein the iron compound is iron oxide.

26. The method according to claim 23, wherein the alkali metal compound is a potassium compound.

27. The method according to claim 23, wherein the catalyst further comprises potassium ferrite.

28. The method according to claim 23, wherein conditions effective to dehydrogenate at least a portion of said alkylaromatic hydrocarbons to produce alkenylaromatic hydrocarbons include a temperature of from 540° C. to 660° C., a pressure of from 0.3 atm to 1.5 atm, and a LHSV of from 0.1 hr⁻¹ to 5 hr⁻¹.
